(19) United States
(12) Patent Application Publication
(10) Pub. No.: US 2014/0274773 A1
(43) Pub. Date: Sep. 18, 2014

(54) SYSTEMS AND METHODS FOR EMPLOYING PODOCALYXIN AND TRA HUMAN STEM CELL MARKERS AS PROGNOSTIC MARKERS FOR AGGRESSIVE AND METASTATIC CANCER

(71) Applicant: WILLIAM MICHAEL SCHOPPERLE, South Boston, MA (US)

(72) Inventor: WILLIAM MICHAEL SCHOPPERLE, South Boston, MA (US)

(21) Appl. No.: 14/206,551

(22) Filed: Mar. 12, 2014

Related U.S. Application Data
(60) Provisional application No. 61/782,087, filed on Mar. 14, 2013.
(60) Provisional application No. 61/782,125, filed on Mar. 14, 2013.

(51) Int. Cl. G01N 33/574; C12Q 1/68

(52) U.S. Cl. CPC G01N 33/57492; G01N 33/57434; C12Q 1/6886
USPC 506/9; 435/287.2; 506/16; 435/6.12; 435/6.11; 435/7.23

(57) ABSTRACT

The invention relates to systems and methods to detect podocalyxin-expressing and TRA-expressing cancer stem cells or cancer cells with stem cell-like properties in a tissue cell sample obtained from a localized tumor of a patient, and to determine a diagnosis or prognosis of aggressive and metastatic cancer by employing podocalyxin and TRA human stem cell markers as prognostic markers.

### CROSS REFERENCE TO RELATED PATENT APPLICATIONS

This patent application claims the benefit of priority under 35 U.S.C. §119(e) from U.S. Provisional Patent Application No. 61/782,087 entitled “Systems and Methods for Employing TRA Human Stem Cell Marker as Prognostic Marker for Aggressive and Metastatic Cancer”, filed on Mar. 14, 2013; and U.S. Provisional Patent Application No. 61/782,125 entitled “Systems and Methods for Employing Podocalyxin Human Stem Cell Marker as Prognostic Marker for Aggressive and Metastatic Cancer”, filed on Mar. 14, 2013; which are incorporated in their entirety herein by reference.

### FIELD OF THE INVENTION

The invention relates to systems and methods to detect podocalyxin-expressing and TRA-expressing cancer stem cells or cancer cells with stem cell-like properties in a tissue cell sample obtained from a localized tumor, and to determine a diagnosis or prognosis of aggressive and/or metastatic cancer by employing podocalyxin and TRA human stem cell markers as prognostic markers.

### BACKGROUND OF THE INVENTION

Most solid-tumor human cancers can be broadly categorized into two general types, localized cancer and aggressive/metastatic cancer. Localized cancer does not spread, is generally non-lethal, and is usually manageable and treatable with current standard cancer therapies. In contrast, aggressive cancer generally spreads to other parts of the body, usually is not curable with conventional cancer therapies, and is almost always lethal for the patient. One of the hypothesis of the stem cell theory of cancer is that aggressive and metastatic cancer is driven by cancer stem cells derived from normal adult stem cells which reside within the body. Another hypothesis is that localized cancer cells transform into aggressive cancer cells through the acquisition of specialized stem-cell functions allowing them to detach from the primary tumor, move through surrounding tissues and into the blood supply and finally, to reattach and seed new tumor growth elsewhere in the body.

The time course for progression from localized cancer to lethal aggressive/metastatic cancer has significant variability. Some cancers progress rapidly while others remain indolent for years prior to progressing to an aggressive/metastatic form, and some cancers will never progress to an aggressive/metastatic form. It is not known why some cancers progress rapidly to an aggressive/metastatic form while others do not. For example, annually in the United States, approximately 240,000 men will be diagnosed with localized prostate cancer and studies have estimated that only about 10% of the hundreds of thousands of men diagnosed with localized prostate cancer will actually develop the lethal aggressive/metastatic form of the disease.

The standard protocol for diagnosing localized cancer is a biopsy. Several tissue cores are harvested from the a target area and are histologically examined by a pathologist. Abnormal cells may be detected based on cell morphology. For example, when diagnosing prostate cancer, if there are abnormal-looking cells identified in the tissue cores, the tissue is given a Gleason score which is a numerical rating of how undifferentiated are the abnormal-looking cells as compared with normal cells. Typically, the Gleason score ranges from Gleason 6 which is the least undifferentiated tissue as compared to normal tissue (this is diagnosed as the lowest grade/least aggressive prostate cancer) to Gleason 10 which is the most undifferentiated tissue (this is diagnosed as the highest grade/most aggressive cancer). In general, the higher the Gleason score, the more likely the patient will develop an aggressive/metastatic form of cancer. However, the Gleason score is not necessarily accurate for all cancer patients. For example, it has been demonstrated that patients with Gleason 6 localized cancer may develop the aggressive disease while patients with Gleason 10 localized cancer may not progress beyond localized cancer.

Typical treatment for patients diagnosed with cancer is surgery and/or radiation. It is known that there are serious complications associated with these cancer therapies. It is also known that not all patients who are diagnosed with cancer, e.g., the localized form, will develop the aggressive form of cancer. Thus, there is growing concern that many cases of cancer are incorrectly diagnosed and there are patients undergoing treatments that are not necessary. For example, new clinical studies have revealed that the current test for diagnosing prostate cancer (Gleason score and PSA blood test) has little effect on the annual death rate from prostate cancer as compared with no screening at all.

There is a tremendous need for new and better diagnostic and prognostic clinical tests to determine which cancer patients have aggressive, deadly cancer and therefore should be treated, and which patients have a localized, non-threatening form of cancer (or no cancer at all) and therefore should not be treated, but rather actively monitored for any biological changes within the localized tissue.

Research studies have suggested that the aggressive/metastatic and lethal forms of cancer are caused by a unique type of cancer cells which have stem cell-like properties. These cancer stem cells or cancer cells with unique stem cell-like properties can develop in localized cancer tissue. It is believed that these cancer stem cells make up only a small portion of the tumor bulk, but they have stem cell-like properties and it is believed that these are the cells which are highly resistant to current therapies, are highly motile, can re-seed the tumor and can cause new growth in other parts of the body (e.g., metastasize).

The development of a new clinical test capable of detecting the presence of aggressive cancer stem cells within the localized cancer or tissue may have the potential to determine which patients have aggressive and deadly cancer and therefore, should be treated and which patients have localized and non-threatening cancer (or no cancer at all) and therefore, should not be treated but rather actively monitored for any biological changes within the localized tissue.

Podocalyxin is a cell surface plasma membrane protein which is highly expressed on the surface of human pluripotent stem cells, such as human embryonic stem cells, and is a human stem cell-defining marker for human embryonic stem cells. Pluripotent stem cells are fundamental stem cells having biological properties to differentiate into any one of the two hundred different kinds of cells that make up the human body. Podocalyxin has several putative cellular functions which include a cellular adhesion function, an anti-adhesion protein and a cell motility function. Thus, these functions of podocalyxin expressed on cancer stem cells may allow the cancer cells to survive within a patient and furthermore, to spread or metastasize and also re-seed the tumor after standard cancer therapy treatments.

The detection or identification of podocalyxin on certain cancer cells within localized cancer or within tissue may be used to identify aggressive cancer stem cells or aggressive cancer cells with stem cell-like properties.

TRA is a carbohydrate (sugar) molecular structure which is present or expressed in large or very large amounts on the cell surface of undifferentiated human embryonic pluripotent stem cells. This carbohydrate molecule was first discovered on human embryonal carcinoma pluripotent cancer stem cells by using two distinct mouse monoclonal antibodies: TRA-1-60 and TRA-1-81. At the time of discovery, the epitopes to which these monoclonal antibodies bind were unknown and therefore, the epitopes where named after the antibodies. The TRA-1-60 and TRA-1-81 monoclonal antibodies are widely used in stem cell research to characterize normal human embryonic stem cells. The TRA epitope carbohydrate structure has been identified as a pluripotency associated Type 1 lactosamine epitope. It has also been reported that the minimal epitope necessary for binding of the TRA-1-60 and TRA-1-81 antibodies is Galβ1-3GlcNAcβ1-3Galβ1-4GlcNAc on an extended tetrasaccharide mucin type O-glycan structure in human embryonic stem cells with a likely structure Galβ1-3GlcNAcβ1-3Galβ1-4GlcNAcβ1-6(Galβ1-3)GalNAc.

It is desirous to develop systems and methods for detecting the presence of cancer stem cells or stem cell-like cancer cells in a tumor which have specialized stem cell-like properties enabling them to grow and metastasize throughout the body resulting in a lethal form of the disease. The systems and methods of the invention include the diagnosis and prognosis of localized tumors based on the presence or absence of cancer stem cells or stem cell-like cancer cells that express podocalyxin and/or TRA in the localized tumor. Further, based on the diagnosis and prognosis, the systems and methods of the invention include selecting and implementing an effective therapy and treatment regimen.

### SUMMARY OF THE INVENTION

In one aspect, the invention provides a system to determine a diagnosis or prognosis of aggressive cancer cells in a patient having a localized tumor. The system includes a biopsy means to obtain a tissue cell sample from the localized tumor; a detecting means to determine the presence of podocalyxin and/or TRA expressed on cancer stem cells in the tissue cell sample; an indicator means to display the presence or absence of TRA-expressing cancer stem cells in the tissue cell sample; a medical diagnosis or prognosis of aggressive or metastatic cancer cells in the patient based on the presence of podocalyxin-expressing and/or TRA-expressing cancer stem cells in the tissue cell sample; and a medical diagnosis or prognosis of non-aggressive and localized cancer cells in the patient based on the absence of podocalyxin-expressing and/or TRA-expressing cancer stem cells in the tissue cell sample.

The detecting means can be selected from the group consisting of immunoassays, immunostaining, immunofluorescence, immunohistochemistry, direct IHC, indirect IHC, immunocytochemistry, in situ hybridization, fluorescent ISH, FISH in suspension, western blot, flow cytometry, fluorescence-activated cell sorting, imageStream, turtle probes, target primed rolling circle PRINS, luminex assay, polymerase chain reaction, and mass spectrometry.

In another aspect, the invention provides a method of determining a diagnosis or prognosis of aggressive cancer cells in a first patient having a localized tumor. The method includes obtaining a first tissue cell sample from the localized tumor; evaluating the first tissue cell sample for the presence of podocalyxin and/or TRA expressed on cancer stem cells; determining a presence or an absence of podocalyxin-expressing and/or TRA-expressing cancer stem cells in the first tissue cell sample; providing a positive medical diagnosis or prognosis of aggressive cancer cells for the first patient based on the presence of the podocalyxin-expressing and/or TRA-expressing cancer stem cells in the first tissue cell sample; and providing a negative medical diagnosis or prognosis of aggressive cancer cells for the first patient based on the absence of the podocalyxin-expressing and/or TRA-expressing cancer stem cells in the first tissue cell sample.

The first tissue cell sample can be compared to a second tissue cell sample selected from the group consisting of an earlier obtained tissue cell sample from the first patient or a control tissue cell sample obtained from a second patient without cancer.

The first tissue cell sample can be compared to a predetermined threshold value.

The method can further include providing a medical diagnosis of the presence of aggressive cancer cells in the first patient based on a level of the podocalyxin-expressing and/or TRA-expressing cancer stem cells in the first tissue cell sample being measurably greater than a level of podocalyxin-expressing and/or TRA-expressing cancer stem cells in the second tissue cell sample or providing a medical diagnosis of the absence of aggressive cancer cells in the first patient based on a level of the podocalyxin-expressing and/or TRA-expressing cancer stem cells in the first tissue cell sample being essentially equal to or measurably less than a level of podocalyxin-expressing and/or TRA-expressing cancer stem cells in the second tissue cell sample.

The method can further include providing a medical diagnosis of the presence of aggressive cancer cells in the first patient based on a level of the podocalyxin-expressing and/or TRA-expressing cancer stem cells in the first tissue cell sample being essentially equal to or measurably greater than the threshold value or providing a medical diagnosis of the absence of aggressive cancer cells in the first patient based on a level of the podocalyxin-expressing and/or TRA-expressing cancer stem cells in the first tissue cell sample being measurably less than the threshold value.

In yet another aspect, the invention provides a method of selecting a medical treatment for a patient having a localized tumor. The method includes obtaining a tissue cell sample from the localized tumor; analyzing the sample for a presence of podocalyxin-expressing and/or TRA-expressing cancer stem cells selected from the group consisting of podocalyxin-specific antibodies and/or TRA-1-60, TRA-1-81 and mixtures thereof; determining the presence or an absence of the podocalyxin-expressing and/or TRA-expressing cancer stem cells in the sample; providing a positive medical diagnosis or prognosis of aggressive cancer for the patient based on the presence of the podocalyxin-expressing and/or TRA-expressing cancer stem cells in the tissue cell sample; and providing a negative medical diagnosis or prognosis of aggressive cancer for the patient based on the absence of the podocalyxin-expressing and/or TRA-expressing cancer stem cells in the tissue cell sample.

The analyzing of the sample can employ a binding material selected to interact with the podcalyxin-expressing and/or TRA-expressing cancer stem cells in the sample. The binding material is an antibody.

In still another aspect, the invention provides a kit for detecting and diagnosing aggressive cancer stem cells in a patient. The kit includes a tool for obtaining a tissue cell sample from the patient; an analyzing means for determining the presence or absence of podocalyxin-expressing and/or TRA-expressing cancer stem cells in the tissue cell sample; and an indicator means to display the presence or absence of the podocalyxin-expressing and/or TRA-expressing cancer stem cells in the sample.

The analyzing means can employ a biotinylated material selected to interact with the podocalyxin-expressing and/or TRA-expressing cancer stem cells in the sample.

In certain embodiments, the localized tumor is a localized prostate tumor.

### DETAILED DESCRIPTION OF THE INVENTION

The invention includes systems and methods for detecting podocalyxin-expressing and TRA-expressing cancer stem cells and determining a diagnosis or prognosis of aggressive and/or metastatic cancer within a localized tumor by employing podocalyxin and/or TRA human stem cell markers as prognostic markers in a tissue cell sample obtained from the localized tumor. Further, the invention includes selecting a regimen of therapy and treatment for the patient based on a positive or negative diagnosis or prognosis of aggressive and/or metastatic cancer.

As used herein and the claims, the terminology “podocalyxin-expressing cancer stem cells” and related variations thereof, means a cell with the podocalyxin protein molecule present on the surface of the cancer stem cell, or podocalyxin could be present intracellularly in the cytoplasm or associated with an organelle, or podocalyxin could be present in the extracellular space and/or the glycocalyx surrounding the cell and not necessarily in direct physical contact with the cancer stem cell. Further, as used herein and the claims, “TRA-expressing cancer stem cells” and related variations thereof, means a cell with the TRA molecule present on the surface of the cancer stem cell, or TRA present intracellularly in the cytoplasm or associated with an organelle, or TRA present in the extracellular space and/or the glycocalyx surrounding the cell and not necessarily in direct physical contact with the cancer stem cell. Furthermore, as used herein and the claims, the terminology “cancer stem cell” and related variations thereof, means a cancer stem cell with the ability to differentiate into other cell types, or a cancer cell with some properties which are shared with stem cells but the cell might not be able to differentiate into other cell types, or a cell which is not defined as cancer by current medical standards but would have cellular changes which would not be present in a normal cell. Moreover, for ease of description, the invention is described herein with respect to a localized prostate tumor and prostate cancer. However, this description is not intended to be limiting. It is contemplated and should be understood that the invention relates to all types of localized cancer tumors and cancer stem cells including, but not limited to, breast tumors and breast cancer.

Podocalyxin is a cell surface plasma membrane adhesion protein which is highly expressed on the surface of human pluripotent stem cells (e.g., human embryonic stem cells). As used herein, the term “normal” refers to the absence of cancer. Pluripotent stem cells are fundamental stem cells having biological properties to differentiate into any one of the two hundred different kinds of cells that make up the human body. TRA is an embryonic carbohydrate structure which is present on the cell surface of undifferentiated human embryonic stem cells and human pluripotent stem cells. It is also present on embryonal carcinoma cancer stem cells which are considered to be a malignant version of the normal embryonic stem cells. The TRA carbohydrate structure is not found or detected in normal adult human tissues. It is believed that the TRA structure is an embryonic specific Type 1 lactosamine oligosaccharide structure. The mouse monoclonal antibodies TRA-1-60 and TRA-1-81 bind specifically to the TRA structure. These antibodies are used to characterize normal human embryonic stem cells and other human pluripotent stem cells.

Without intending to be bound by any particular theory, it is believed that aggressive and metastatic forms of prostate cancer are caused by a unique type of prostate cancer cells which have stem cell-like properties. These cancer stem cells can develop in localized prostate cancer tissue. These cancer stem cells have the podocalyxin and/or TRA structure expressed on their surface. These podocalyxin-expressing and/or TRA-expressing cancer stem cells make up only a small portion of the tumor bulk, but they have stem cell-like properties and it is believed that these cells are highly resistant and motile, can re-seed the tumor and cause new growth elsewhere in the body. Thus, a test to determine if there are any aggressive and/or metastatic cancer stem cells (or cancer cells having stem cell-like properties) in a localized tumor of a patient includes using podocalyxin and/or TRA-specific antibodies (e.g., TRA-1-60, TRA-1-81) and/or any other polyclonal or monoclonal antibodies specific to the podocalyxin and/or TRA structures to detect the presence of any podocalyxin-expressing and/or TRA-expressing cancer stem cells. If podocalyxin and/or TRA reactivity is positive in a sample, e.g., biopsy, of a localized tumor by immunostaining or other methods used to detect the podocalyxin and/or TRA antibodies, this result indicates that there are aggressive and/or metastatic cancer stem cells in the localized tumor which can eventually lead to the aggressive and/or metastatic form of the cancer and therefore, the patient is administered aggressive treatment, such as surgery, chemotherapy, radiation therapy or combinations thereof. If no positive podocalyxin and/or TRA reactivity is detected, this result indicates that the localized tumor is not aggressive and therefore, no aggressive treatment is needed for the patient and subsequently, the patient's condition and progress may simply be monitored and assessed. This test can be particularly useful for determining the treatment for prostate cancer patients with localized disease who have Gleason scores in the range of 6 to 8 or 7 to 8 because it is often difficult to determine the need for aggressive or non-aggressive treatment for these mid-range scores. That is, less aggressive or non-aggressive treatment is typically considered appropriate for Gleason scores of 5 or less, and for Gleason scores of 9 or 10 more aggressive treatment is typically considered appropriate. However, there is often an uncertainty as to the appropriate treatment recommendation for Gleason scores of 6, 7 or 8. For these mid-range scores, it is desirable to have additional information and data to contribute to a diagnosis and prognosis of aggressive and/or metastatic cancer. For example, the presence or absence of podocalyxin and/or TRA reactivity can be considered in conjunction with the Gleason score. Thus, for a Gleason score of 6, 7 or 8 and an absence of podocalyxin and/or TRA reactivity, less aggressive or non-aggressive treatment may be appropriate. While, for a Gleason score of 6, 7 or 8 and a presence of podocalyxin and/or TRA reactivity, more aggressive treatment may be appropriate.

It is contemplated that podocalyxin and/or TRA may be present in normal patients at low levels and may be present in patients having aggressive and/or metastatic cancers at higher levels. In accordance with the invention, in certain embodiments, the level of podocalyxin and/or TRA in a tumor sample obtained from a cancer-containing patient can be compared to the level of podocalyxin and/or TRA in a control sample obtained from a non-cancer-containing patient. If the level of podocalyxin and/or TRA in the tumor sample from the cancer-containing patient is measurably greater than or measurably higher than the level of podocalyxin and/or TRA in the control sample, then there is a diagnosis of the presence of podocalyxin-expressing and/or TRA-expressing cancer stem cells in the tumor sample from the cancer patient. Correspondingly, there may be a prognosis of aggressive and/or metastatic cancer in the cancer-containing patient. Further, there may be a recommendation of an aggressive treatment regimen for the cancer-containing patient. In contrast, if the level of podocalyxin and/or TRA in the tumor sample from the cancer-containing patient is measurably less or measurably lower than or essentially equal to the level of podocalyxin and/or TRA in the control sample, then there is a diagnosis of the absence of podocalyxin-expressing and/or TRA-expressing cancer stem cells in the tumor sample from the cancer patient. Correspondingly, there may be a prognosis of no aggressive and/or metastatic cancer in the cancer-containing patient. Further, there may be a recommendation of a less aggressive treatment regimen for the cancer-containing patient.

In certain other embodiments, the level of podocalyxin and/or TRA in a tumor sample obtained from a cancer-containing patient can be compared to the level of podocalyxin and/or TRA in another, e.g., earlier obtained, biological sample from the cancer-containing patient. If the level of podocalyxin and/or TRA in the tumor sample from the cancer-containing patient is measurably greater than or measurably higher than the level of podocalyxin and/or TRA in the other, e.g., earlier obtained, biological sample from the cancer-containing patient, then there is a diagnosis of the presence of podocalyxin-expressing and/or TRA-expressing cancer stem cells in the patient. In contrast, if the level of podocalyxin and/or TRA in the tumor sample of the cancer-containing patient is measurably less than or measurably lower than or essentially equal to the level of podocalyxin and/or TRA in the other, e.g., earlier obtained, biological sample from the cancer-containing patient, then there is a diagnosis of the absence of podocalyxin-expressing and/or TRA-expressing cancer stem cells in the patient.

In other embodiments, the presence or absence of podocalyxin and/or TRA may be determined based on comparison of a tumor sample to a threshold value. There may be a threshold level or value of podocalyxin and/or TRA such that when the level of podocalyxin and/or TRA measured in the tumor sample is measurably greater than or measurably higher than or essentially equal to the threshold level, the presence of podocalyxin-expressing and/or TRA-expressing cancer stem cells is determined and when the level of podocalyxin and/or TRA measured in the tumor sample is measurably less than or measurably lower than the threshold level, the absence of podocalyxin-expressing and/or TRA-expressing cancer stem cells is determined.

In accordance with the invention, in certain embodiments, a threshold level of podocalyxin and/or TRA or a threshold range of levels of podocalyxin and/or TRA found in normal, healthy patients may be determined by obtaining biological samples from these patients and measuring the level of podocalyxin and/or TRA in these patients. Based on this data, a threshold level or threshold range can be established and employed to determine the presence or absence of podocalyxin-expressing and/or TRA-expressing cancer stem cells in tumor samples. When the podocalyxin and/or TRA level in a tumor sample from a cancer-containing patient is measurably above or essentially equal to the threshold level/range of podocalyxin and/or TRA, there is made a diagnosis of the presence of podocalyxin-expressing and/or TRA-expressing cancer stem cells. When the podocalyxin and/or TRA level in a tumor sample from a cancer-containing patient is measurably below the threshold level/range of TRA, there is made a diagnosis of the absence of podocalyxin-expressing and/or TRA-expressing cancer stem cells.

In general, a determination of the presence of (or positive reactivity of) podocalyxin and/or TRA in a tumor sample of a cancer-containing patient corresponds to a prognosis of aggressive and/or metastatic cancer and a recommendation of aggressive treatment for the cancer-containing patient, and a determination of the absence of (or no reactivity of) podocalyxin and/or TRA in a tumor sample of a cancer-containing patient corresponds to a prognosis of non-aggressive and/or no metastatic cancer and a recommendation of less aggressive treatment for the cancer-containing patient. As previously described, aggressive or more aggressive treatment includes surgery, chemotherapy, radiation therapy or combinations thereof and non-aggressive or less aggressive treatment includes simply surveillance and monitoring and assessing the patient.

The systems and methods of the invention have advantages over those conventionally employed for the detection of cancer including, for example, early detection or prognosis of aggressive cancer, and the ability to customize a treatment regimen in accordance with the aggressive nature of the cancer. The collection, testing and evaluation of a biological sample including tissue cores or cells from a localized cancer tumor allows, for aggressive cells to be detected and a diagnosis or prognosis made prior to the formation of aggressive or metastatic tumors. The removal of tissue or cells can be performed by known biopsy and surgery techniques and tools. The analysis of the biological sample is performed accordance with conventional techniques that are known to reduce the risk of obtaining false positive and false negative results.

Further, the systems and methods of this invention allow for an early detection or prognosis of a lack of aggressive cancer progression based on the absence of podocalyxin-expressing and/or TRA-expressing cancer stem cells, such that a treatment regimen can be customized for the patient. For example, the patient can be merely placed on active surveillance and monitoring, without the need to be subjected to radiation, chemotherapy, surgery, or combinations thereof. Thus, reducing or precluding unnecessary treatments and the potential serious complications associated therewith.

The tissue or cell sample can be collected in a labeled container or placed in a labeled container following collection. The label of the container may contain a unique identification number and one part of the label may be transferable to a slide. The sample can be collected in a container which may contain stabilizing agents to aid in preservation of the sample and of the signal/marker quality in the sample. Stabilizing agents include but are not limited to pH-buffers, Protease-inhibitors, RNase inhibitors, fixatives and other compounds/components known to persons skilled in the art. Collected samples may be stored at the site of collection at suitable temperature or they may be transported to a local or external laboratory for preparation.

The sample may be processed using various conventional techniques in order to obtain an optimal signal from any aggressive cancer cells present in the sample. Processing the sample may include, but is not limited to, filtration, precipitation, immunoprecipitation, flow-sorting, lyzing, centrifugation, cooling, freezing, heating or any other methods known to a person skilled in the art. Preferably, the sample is treated to allow optimal detection of podocalyxin-expressing and/or TRA-expressing stem cells. This is, for example, performed by treating the sample in a manner that allows the molecules/cells of the sample to remain intact and, as far as possible, also retain their original morphology.

The sample may be analyzed for the presence of biomarkers for the detection of podocalyxin-expressing and/or TRA-expressing cells and aggressive cancer cells using a variety of analyses known in the art. These analyses include immunoassays, immunostaining, immunofluorescence, immunohistochemistry, direct IHC, indirect IHC, immunocytochemistry, in situ hybridization, fluorescent ISH, FISH in suspension, western blot, flow cytometry, fluorescence-activated cell sorting, imageStream, turtle probes, target primed rolling circle PRINS, luminex assay, polymerase chain reaction, mass spectrometry and the like.

In accordance with certain embodiments, analysis results showing positive reactivity is indicative of the presence of podocalyxin-expressing and/or TRA-expressing cells in the sample being analyzed and little or no reactivity is indicative of the absence of podocalyxin-expressing and/or TRA-expressing cells in the sample being analyzed.

In certain embodiments, the systems and methods in accordance with the invention are effective to sample localized prostate tumors, determine the presence or absence of podocalyxin-expressing and/or TRA-expressing cells in the sample and provide a diagnosis or prognosis of aggressive/metastatic prostate cancer for the patient. The systems and methods described herein are equally applicable to tumors in other organs or tissues in the body. For example, and without limitation, the ovary, colon, breast, and testis.

Further, immunostaining and/or enzymatic assays for the detection of the at least one biochemical marker can be used during the screening for cancer cells in the sample. Furthermore, in certain embodiments, analyzing the tissue cell samples includes the use of a binding material which is selected to interact with podocalyxin-expressing and/or TRA-expressing cancer stem cells. The binding material may be effective to separate and isolate the podocalyxin-expressing and/or TRA-expressing cancer stem cells from the biological sample. Suitable binding materials are known in the art or may be prepared. In accordance with certain embodiments, the binding material is an antibody and may be biotinylated.

In certain embodiments, in accordance with the invention, tissue or cell samples are collected from patients with localized cancer and patients with metastatic cancer. The samples collected from these two groups of patients are evaluated to determine the presence or absence of podocalyxin-expressing and/or TRA-expressing cancer stem cells. The levels of podocalyxin and/or TRA in the samples are measured by standard methods (e.g., elisa assays, western blotting and the like) using the podocalyxin-specific antibodies and/or TRA-1-60 and TRA-1-81 monoclonal antibodies. These antibodies are commercially available from numerous sources and are widely used by the human stem cell research community for the characterization of embryonic stem cells. The levels of podocalyxin and/or TRA in the samples of patients having cancer (localized and metastatic) are compared with each other, and are compared with the healthy control samples.

Each of the podocalyxin and TRA carbohydrate structure is a marker of human embryonic stem cells. As previously indicated, there may be low levels of podocalyxin and/or TRA in a healthy individual due to the presence of podocalyxin and/or TRA on normal stem cells such as embryonic stem cells. However, in a disease state, the levels of podocalyxin and/or TRA may be increased as compared to the levels present in the healthy/normal body.

In certain embodiments as above-described, the invention is used for the detection and diagnosis of aggressive prostate cancer in male patients. In other embodiments, this invention is used for the detection and diagnosis of other aggressive cancers in both male and female patients.

In an aspect, the invention provides a system for detecting the presence or absence of podocalyxin-expressing and/or TRA-expressing cancer cells in a tissue cell sample and a diagnosis of the presence or absence of aggressive and/or metastatic cancer stem cells in a patient. The system can include a means or mechanism, e.g., biopsy tool, for obtaining the tissue cell sample from the patient. Suitable biopsy tools and biopsy procedures are well known in the art. The system further includes a means or mechanism for detecting podocalyxin-expressing and/or TRA-expressing cancer cells and determining their presence or absence in the sample. In certain embodiments, a detector means is employed to determine the presence or absence of anti-podocalyxin, TRA-1-60 and/or TRA-1-81 in the sample. Furthermore, the device includes an indicator means or mechanism for converting and/or transmitting the result of the detector means, e.g., the presence or absence of anti-podocalyxin, TRA-1-60 and/or TRA-1-81 in the sample, to a displayable output to be read and/or interpreted by the patient and/or a medical professional to provide a medical diagnosis of aggressive, e.g., metastatic, or non-aggressive, e.g., localized, cancer cells.

In certain embodiments, the system can include a kit, e.g., test kit. The test kit can include a device or mechanism for obtaining a tissue cell sample from a patient, staining the sample, evaluating the results of the stained sample to determine the presence of podocalyxin-expressing and/or TRA-expressing cancer stem cells, and providing a diagnosis of aggressive or metastatic cancer in the patient. The kit can be employed by a trained professional, e.g., a physician, nurse, physician assistant or medical technician. It will be appreciated that kits in accordance with the invention may include test kit designs and configurations known in the art or modifications thereto. As is typical with test kits, a standard key or chart may be developed to correlate and interpret the results. For example, in evaluating the stained tissue cell sample, the level or intensity of staining observed can be correlated with the presence or reactivity of podocalyxin and/or TRA. No staining is typically indicative of an absence or no reactivity of podocalyxin and/or TRA. The observation of staining may be further characterized as slight, moderate, strong and very strong. The increase in intensity of the staining can correlate with the increased presence, level or intensity or reactivity of the podocalyxin and/or TRA in the sample and therefore, the increased presence of cancer stem cells or stem cell-like cells in the sample.

In certain embodiments, the TRA-1-60 and TRA-1-81 staining assay may be developed as a kit to be used in clinical labs both in hospitals and independent commercial labs. The kit can include primary antibodies specific to TRA-1-81 and TRA-1-60, conjugated secondary antibodies specific for the primary antibodies, buffers and signaling substrate for assay read out. The kit can be used to determine the level or intensity of expression of TRA-1-60 and TRA-1-81 in cancer biopsies obtained from cancer-containing patients and therefore, to determine the level or intensity of embryonic and pluripotent characteristics and properties of the cancer.

The systems and methods of the invention may be used to determine the presence or absence or level of cancer stem cells or cancer cells having stem cell-like properties, how aggressive is the cancer in a patient and the likelihood that localized cancer will metastasize or develop into aggressive cancer. The results obtained by the systems and methods of the invention provide a source of information and data for consideration in making these determinations. As previously indicated, cancer in patients is often categorized according to a Gleason rating. It is typical for practitioners to correlate aggressive cancer with a high Gleason score and non-aggressive cancer with a low Gleason score. However, it has been found that cancer having a high Gleason score does not necessarily metastasize nor develop into aggressive cancer and it has also been found that cancer having a low Gleason scores can, in fact, metastasize and develop into aggressive cancer. Thus, reliance or dependence solely on Gleason rating does not result in accurate and consistent diagnoses and prognoses of cancer in patients. Therefore, it is believed that the information and data provided by the systems and methods of the invention contribute to increasing the accuracy and consistency of determining and assessing cancer in patients and the progression of the cancer.

In accordance with the invention, the presence or absence of podocalyxin and/or TRA reactivity in a tumor tissue sample can provide information relating to the presence or absence of cancer stem cells or stem-like cells, the likelihood that localized cancer may progress to aggressive and metastasized cancer and, the appropriateness of prescribing aggressive or non-aggressive treatment. For example, as indicated herein, the presence of podocalyxin and/or TRA reactivity may result in a diagnosis of a high likelihood that localized cancer may progress to aggressive and/or metastatic cancer and therefore, a recommendation for administering aggressive treatment. However, as indicated herein, the absence of podocalyxin and/or TRA reactivity may result in a diagnosis of a low likelihood that localized cancer may progress to aggressive and metastatic cancer and therefore, a recommendation for prescribing less aggressive treatment.

The information and data derived from the systems and methods of the invention may be considered alone or in combination with known information and data, such as but not limited to Gleason rating. For example, a patient may be inflicted with cancer having a Gleason score of 7 and typically, the likelihood of progression to aggressive and/or metastatic cancer may be difficult to diagnosis. In these instances, the Gleason score can be combined with the podocalyxin and/or TRA reactivity results to determine the likelihood of the cancer progressing to aggressive and/or metastatic cancer. For example, a Gleason score of 7 in combination with the presence of podocalyxin or TRA, may indicate a high likelihood of progression of the cancer to aggressive and/or metastatic cancer. While a Gleason score of 7 in combination with the absence of podocalyxin or TRA may indicate a low likelihood of progression of the cancer to aggressive and/or metastatic cancer. Further, the Gleason score of 7 in combination with the presence of podocalyxin and the presence of TRA may provide an even higher likelihood of aggressive and metastatic cancer and similarly, the Gleason score of 7 in combination with the absence of podocalyxin and the absence of TRA may provide an even lower likelihood of aggressive and metastatic cancer.

The determination of the likelihood of aggressive and metastatic cancer and therefore, the treatment regimen to be administered may require that medical judgments be made based on various data and information from various sources as described above. In certain embodiments, trained medical personal, such as a physician, may be appropriate to review and interpret the data and information in order to determine the proper treatment for a cancer patient.

For example, in prostate cancer, it is known that patients are often over diagnosed with prostate cancer leading to unnecessary medical procedures having serious complications. In an attempt to reduce over diagnosis of prostate cancer, physicians may place patients with a Gleason score of 5 or 6 on “active surveillance” and monitor the progress of the cancer, whereas patients with Gleason scores of 8, 9 or 10 are candidates for surgery or radiation or both. There is, however, as previously described, uncertainty as to whether patients with Gleason 7 belong in the “watch or treat’ stratification. Knowledge of the level of the TRA-1-60 or TRA-1-81 stem cell markers of a Gleason 7 prostate tumor may be an indication of how aggressive and how likely the tumor with progress to an advance and metastatic state. This type of analysis of primary tumors for pluripotent and embryonic stem cell properties could also be applied to the other Gleason scores. For example, a primary prostate tumor with a Gleason 6 score but with high expression of the TRA-1-60 and TRA-1-81 stem cell markers may be an indication that this is an aggressive cancer which needs to be treated. Likewise, a primary tumor with a Gleason 8 score but with an absence or low expression of TRA-1-60 or TRA-1-81 may be an indication that this patient could benefit from monitoring and “active surveillance”, and no further treatments.

In other examples, patients may be over diagnosed with breast cancer and undergo unnecessary medical treatments. IHC staining of the breast cancer biopsy may be conducted to determine the level of expression of TRA-1-60 and TRA-1-81 in the cancer tissue and to determine the aggressive and metastatic potential of the primary cancer and this information could be used to determine the treatment approach for that patient, regardless of a high or low Gleason rating.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof. Furthermore, the following examples are meant to be illustrative of certain embodiments of the invention and are not limiting as to the scope of the invention.

### Example

To measure the expression level of podocalyxin and the human pluripotent stem cell markers, TRA-1-60 and TRA-1-81, an immunohistochemistry (IHC) technique was used and IHC staining was employed for formalin-fixed, paraffin-embedded human biopsy tissue that was cut into 4 microns thick sections and fixed to microscope glass slides. IHC was conducted on normal tissues, primary cancers (disease confined to organ/no metastases) and metastatic cancers. Slides of formalin-fixed and paraffin-embedded human tissues were obtained from Folio Biosciences (1476 Manning Parkway, Powell, Ohio, 43065) a commercial provider of human biospecimens. In addition, Tissue Microarray slides (TMA) of formalin-fixed and paraffin-embedded human tissues were purchased from Origene (Rockville, Md., catalog number CT565858). Podocalyxin IHC staining was done on normal human prostate tissue, primary prostate cancer tissue and metastatic prostate cancer. TRA-1-60 and TRA-1-81 IHC staining was done on normal tissue, primary cancer and metastatic cancer for prostate, breast, colon, lung, brain and stomach.

To conduct IHC staining for podocalyxin, TRA-1-60 and TRA-1-81, all slides of the various normal and cancer sections were fixed in 10% buffered formalin for 24 hours, sectioned at 5 microns and placed on positively charged slides. Before staining, the slides were dried at 60 C.° overnight. After deparaffinized in Xylene (Fisher Scientific, catalog number X3P-1GAL), the sections were rehydrated through 100% Ethanol (Eki-Chem, catalog number 4085-1GL), 95% Ethanol and 70% Ethanol (Eki-Chem, catalog number 4089-1GL). The sections were then immersed into 1× Diva Decloaker (Biocare Medical, catalog number DV2004LX), and cooked with Decloaking chamber (Biocare Medical, Model: NxGen). For prostate tissues, the sections were cooked at 110 C.° for 5 minutes. For other tissues, the sections were cooked at 95 C.° for 40 minutes. The sections were then blocked by BLOXALL (Vector labs, catalog number SP-6000), Streptavidin/Biotin Blocking kit (Vector labs, catalog number SP-2002) and Background Terminator (Biocare Medical, catalog number BT967L). The primary antibody TRA-1-60 (1:500 working dilution, Life Technologies, catalog number 411000), TRA-1-81 (1:500 working antibody dilution, Life Technologies, catalog number 411100) and podocalyxin (1:500 working antibody dilution, Life Technologies, catalog number 393800) were applied to each slide with 10% normal goat serum (Southern Biotech, catalog number 0060-01) in PBS, and incubated overnight at 4 C.°. After washing by PBS (Fisher Scientific, catalog number BP2944-100), biotinylated anti-mouse IgM (Vector labs, catalog number BA-2020) or biotinylated anti-mouse IgG (Vector labs, catalog number BA-9200) was applied at 1:500 for 1 hour at room temperature. Then the slides were incubated with Streptavidin-HRP (Vector labs, catalog number SA-5704) for 30 minutes at room temperature, followed by DAB (Vector labs, catalog number SK4105) color reaction. The slides were counterstained with hematoxylin (Vector labs, catalog number H-3404). Between the incubations of different reagents or antibodies, the slides were washed by PBS (Fisher Scientific, catalog number BP2944-100). Negative control tissue sections were conducted in the absence of the primary antibodies listed above, in parallel of all assays to control for background staining.

After IHC staining was performed on the slides of tissues, the level of IHC staining of the three pluripotent stem cell markers, podocalyxin, TRA-1-60 and TRA-1-81 were analyzed with an Evos xl core light Microscope (Advance Microscopy Group). An IHC staining score system was developed and used based on staining intensity of the tissues to measure overall staining of the slides. The control IHC staining was done with no primary antibodies to set the level of 0 staining.
Staining Score 0: No staining;
Staining Score 1: A few positive cells in whole slides;
Staining Score 2: Moderate staining of tissues;
Staining Score 3: Strong staining of tissues; and
Staining Score 4: Very strong and intense staining of all tissue (this score was only used for podocalyxin staining with prostate cancer).

### Results of Podocalyxin IHC Staining on Prostate Cancer

The results obtained of the staining are shown in Table 1. Podocalyxin IHC staining was done on six normal prostate samples, seventeen primary prostate cancers (disease localized to prostate gland with no metastasis present and with Gleason scores ranging from 6 to 9) and five metastatic prostate cancers. A matching control slide with no primary podocalyxin antibody was done for each sample. Positive podocalyxin staining could be detected in the blood vessels within the normal and cancer prostate tissues for all samples. This result was expected since normal vascular endothelial cells have been shown to express podocalyxin. No podocalyxin staining was detected in six of six normal prostate samples. All primary and metastatic prostate cancer samples had podocalyxin staining (staining score >0). In the primary prostate cancers with a Gleason score of either 6 or 7, eleven out of thirteen samples had a strong staining score (score of 3 or 4) and the staining score mean value for the thirteen samples was 2.69. In the primary prostate cancers with a Gleason score of 8 or 9, three of four samples had a strong staining score and a mean value of 3.25 for the group. Five out of five metastatic prostate samples had a strong staining score and a mean value staining score of 3.8. All 28 control slides had no podocalyxin staining.

The results of the IHC podocalyxin staining in prostate tissues showed that podocalyxin is not expressed in normal prostate cells, but is expressed in primary and metastatic prostate cancers. Furthermore, the intensity of podocalyxin staining increased with level of aggressiveness of the prostate cancer. The Gleason score is a cell morphology based score of a prostate biopsy determined by a pathologist with a range of 0 (for normal prostate tissue) to 5 (for most undifferentiated and abnormal prostate tissue). The Gleason score is the sum of two of the most common patterns of tissue seen in the biopsy and typically in clinical practice today the Gleason score ranges from 5 to 10 for the presence of cancer in the prostate biopsy with a Gleason 5 score being the most differentiated and normal looking prostate cancer tissue (and the least aggressive type of primary cancer) and a Gleason score of 10 being the least differentiated and most abnormal looking prostate cancer tissue (and the most aggressive type of primary cancer). These podocalyxin IHC results showed that the intensity of podocalyxin staining correlated with an increase in Gleason score.

### Results of TRA-1-60 and TRA-1-81 IHC Staining on Normal Prostate Tissue, Primary Prostate Cancer and Metastatic Prostate Cancer

The staining scoring results obtained are shown in Table 2. The TRA-1-60 and TRA-1-81 antibodies showed similar IHC staining patterns in all prostate tissue tested. The similar staining pattern of the two separate monoclonal antibodies was expected because both antibodies bind to the same pluripotent specific carbohydrate molecule that is expressed on a stem-cell version of podocalyxin which is present in all pluripotent stem cells. In all normal prostate tissue tested (four out of four) there was no positive staining of TRA-1-60 or TRA-1-81 (IHC staining score 0). In the seventeen primary cancers, thirteen out of seventeen samples scored positive with TRA-1-60 and TRA-1-81 staining. In the metastatic prostate cancer tissues, four out of five samples scored very strong staining of three and one out of five metastatic prostate cancer tissues had a moderate staining score of 2. A matching control slide without any primary TRA-1-60 or TRA-1-81 antibody was done for each prostate sample and no staining was detected in any matching control samples.

TABLE 1
Table 1 IHC staining scores of TRA-1-60, TRA-1-81 and
podocalyxin in normal prostate, and in primary and
metastatic prostate cancer tissue samples.

| | Gleason | TRA-1-60 | TRA-1-81 | Podocalyxin |
|---|---|---|---|---|
| Tissue | score | score | score | score |
| Primary prostate cancer | 7 | 2 | 2 | 3 |
| #1 | | | | |
| Primary prostate cancer | 7 | 1 | 1 | 3 |
| #2 | | | | |
| Primary prostate cancer | 7 | 1 | 1 | 3 |
| #3 | | | | |
| Primary prostate cancer | 9 | 0 | 0 | 3 |
| #4 | | | | |
| Primary prostate cancer | 9 | 2 | 2 | 4 |
| #5 | | | | |
| Primary prostate cancer | 8 | 3 | 3 | 4 |
| #6 | | | | |
| Primary prostate cancer | 7 | 0 | 0 | 3 |
| #7 | | | | |
| Primary prostate cancer | 7 | 1 | 1 | 2 |
| #8 | | | | |
| Primary prostate cancer | 7 | 3 | 3 | 3 |
| #9 | | | | |
| Primary prostate cancer | 7 | 2 | 2 | 3 |
| #10 | | | | |
| Primary prostate cancer | 7 | 1 | 1 | 4 |
| #11 | | | | |
| Primary prostate cancer | 7 | 1 | 1 | 3 |
| #12 | | | | |
| Primary prostate cancer | 6 | 1 | 1 | 3 |
| #13 | | | | |
| Primary prostate cancer | 7 | 0 | 0 | 2 |
| #14 | | | | |
| Primary prostate cancer | 7 | 1 | 1 | 2 |
| #15 | | | | |
| Primary prostate cancer | 7 | 0 | 0 | 2 |
| #16 | | | | |
| Primary prostate cancer | 9 | 2 | 2 | 2 |
| #17 | | | | |
| Metastatic prostate | NA | 3 | 3 | 4 |
| cancer | | | | |
| Metastatic prostate | NA | 3 | 3 | 4 |
| cancer | | | | |
| Metastatic prostate | NA | 3 | 3 | 4 |
| cancer | | | | |
| Metastatic prostate | NA | 3 | 3 | 4 |
| cancer | | | | |
| Metastatic prostate | NA | 2 | 2 | 3 |
| cancer | | | | |
| Normal prostate | NA | 0 | 0 | 0 |
| Normal prostate | NA | 0 | 0 | 0 |
| Normal prostate | NA | 0 | 0 | 0 |
| Normal prostate | NA | 0 | 0 | 0 |

All seventeen primary prostate cancer tissues were obtained from Folio Biosciences which provided a Gleason score for each primary prostate cancer. The Gleason score is a cell morphology based score of a prostate biopsy determined by a pathologist with a range of 0 (for normal prostate tissue) to 5 (for most undifferentiated and abnormal prostate tissue). The Gleason score is the sum of two of the most common patterns of tissue seen in the biopsy and typically in clinical practice today the Gleason score ranges from 5 to 10 for the presence of cancer in the prostate biopsy with a Gleason 5 score being the most differentiated and normal looking prostate cancer tissue and a Gleason score of 10 being the least differentiated and most abnormal looking prostate cancer tissue. In the primary prostate cancer tissues that had a Gleason score of 8 or 9, three out of the four tissue samples stained moderate to strong for TRA-1-60 and TRA-1-81. The mean value IHC staining score for TRA-1-60 and TRA-1-81 in the localized/primary prostate cancer tissues with a Gleason score of 8 or 9 was 1.75. In the primary prostate cancer tissues that had a Gleason score of 6 or 7, three out of thirteen tissue samples stained moderate or stronger (staining score of 2 and 3) and ten out of thirteen tissue samples had a weak staining score of 1. The mean value of the IHC staining score for TRA-1-60 and TRA-1-81 in the localized/primary prostate cancer tissues that had a Gleason score of 6 or 7 was 1.08. The results show that the IHC staining intensity of TRA-1-60 and TRA-1-81 correlated with the progression of the Gleason score of the prostate cancer tissue. Furthermore, three out of thirteen localized/primary prostate cancer tissue with the less aggressive prostate cancer Gleason score of 6 or 7 had moderate to strong TRA-1-60 and TRA-1-81 IHC staining that is seen in the high Gleason score prostate cancers and the metastatic prostate cancers.

### Results of TRA-1-60 and TRA-1-81 IHC Staining on Normal Breast Tissue, Primary Breast Cancer and Metastatic Breast Cancer

The TRA-1-60 and TRA-1-81 antibodies showed similar IHC staining patterns in all breast tissue tested. The similar staining pattern of the two separate monoclonal antibodies was expected because both antibodies bind to the same pluripotent specific carbohydrate molecule that is expressed on a stem-cell version of podocalyxin which is present in all pluripotent stem cells. TRA-1-60 and TRA-1-81 IHC staining was negative (staining score of 0) on three out of three normal breast tissue samples. In the localized/primary breast cancer tissues, three out of ten had a 0 staining score, five out of ten had a staining score of 1 and two out of ten had a staining score of 2 for TRA-1-60 and TRA-1-81. For the metastatic breast cancer tissue samples, five out of ten had a TRA-1-60 and TRA-1-81 staining score of 1 and three out of ten had a staining score of 2 while two out of ten had no detectable staining. A matching control slide without any primary TRA-1-60 or TRA-1-81 antibody was done for each breast sample and no staining was detected in any matching control samples.

### Results of TRA-1-60 and TRA-1-81 IHC Staining on Normal Lung, and Primary and Metastatic Lung Cancer

The TRA-1-60 and TRA-1-81 antibodies showed similar IHC staining patterns in all lung samples tested. The similar staining pattern of the two separate monoclonal antibodies was expected because both antibodies bind to the same pluripotent specific carbohydrate molecule that is expressed on a stem-cell version of podocalyxin which is present in all pluripotent stem cells. No staining (staining score 0) was detected on three of three normal lung tissues. A positive TRA-1-60 and TRA-1-81 staining (staining score ≧1) was detected in four of eight primary lung cancers and in two of five metastatic lung cancers. A matching control slide without any primary TRA-1-60 or TRA-1-81 antibody was done for each lung sample and no staining was detected in any matching control samples.

### Results of TRA-1-60 and TRA-1-81 IHC Staining on Normal Colon, and Primary and Metastatic Colon Cancer

The TRA-1-60 and TRA-1-81 antibodies showed similar IHC staining patterns in all colon samples tested. The similar staining pattern of the two separate monoclonal antibodies was expected because both antibodies bind to the same pluripotent specific carbohydrate molecule that is expressed on a stem-cell version of podocalyxin which is present in all pluripotent stem cells. No staining (staining score 0) was detected on two of two normal colon samples. A positive TRA-1-60 and TRA-1-81 staining pattern (staining score ≧1) was seen in one of three primary lung cancers and in three of three metastatic lung cancers. A matching control slide without any primary TRA-1-60 or TRA-1-81 antibody was done for each colon sample and no staining was detected in any matching control samples.

### Results of TRA-1-60 and TRA-1-81 IHC Staining on Metastatic Stomach Cancer

The TRA-1-60 and TRA-1-81 antibodies showed similar IHC staining patterns in all stomach samples tested. Seven out of seven metastatic stomach cancer samples had positive staining (stain score ≧1). A matching control slide without any primary TRA-1-60 or TRA-1-81 antibody was done for each stomach sample and no staining was detected in any matching control samples.

### Results of TRA-1-60 and TRA-1-81 Staining on Brain Cancer

The TRA-1-60 and TRA-1-81 antibodies showed similar IHC staining patterns in all stomach samples tested. Three out of three brain cancers had a positive staining (staining score ≧1). A matching control slide without any primary TRA-1-60 or TRA-1-81 antibody was done for each brain sample and no staining was detected in any matching control samples.

### Results of TRA-1-60 and TRA-1-81 on Normal Tissues

There was no detectable IHC staining on two of two normal endometrial tissues, one of one normal ovary, three of three lymphoid tissues and one of one normal kidney tissue.

Overall, the results showed that the pluripotent and embryonic stem cell markers TRA-1-60 and TRA-1-81 were present in six different cancers including prostate, breast, lung, colon, stomach and brain. The TRA-1-60 and TRA-1-81 stem cell markers are molecular epitopes present on a carbohydrate molecule which are recognized by the TRA-1-60 and TRA-1-81 monoclonal antibodies. These stem cell markers have been shown to be present only on pluripotent or embryonic stem cells and not on differentiated somatic cells. It has been shown that the stem cell markers disappear soon after the initiation of differentiation of the stem cells. The TRA epitope carbohydrate structure has been identified as a pluripotency associated type 1 lactosamine epitope. It has also been reported that the minimal epitope necessary for binding of the TRA-1-60 and TRA-1-81 antibodies is Galβ1-3GlcNAcβ1-3Galβ1-4GlcNAc on an extended tetrasaccharide mucin type O-glycan structure in human embryonic stem cells with a likely structure Galβ1-3GlcNAcβ1-3Galβ1-4GlcNAcβ1-6(Galβ1-3)GalNAc. The TRA carbohydrate structure has been shown to be covalently attached to a stem cell form of the protein podocalyxin which is also highly expressed in pluripotent and embryonic stem cell. The data revealed that human cancer acquires pluripotent and embryonic stem cell properties such as the expression of the TRA-1-60 and TRA-1-81 stem cell markers. The data also revealed that the level of expression of the TRA-1-60 and TRA-1-81 stem cell markers increased in the more aggressive and/or metastatic cancers. Further, the data revealed there was no or very little expression of the TRA-1-60 and TRA-1-81 stem cell markers in normal tissue which was expected since normal tissue does not have embryonic or pluripotent stem cells.

1. A system to determine a diagnosis or prognosis of aggressive cancer cells in a patient having a localized tumor, comprising:
a biopsy means to obtain a tissue cell sample from the localized tumor;
a detecting means to determine the presence of podocalyxin and/or TRA expressed on cancer stem cells in the tissue cell sample;
an indicator means to display the presence or absence of TRA-expressing cancer stem cells in the tissue cell sample;
a medical diagnosis or prognosis of aggressive or metastatic cancer cells in the patient based on the presence of podocalyxin-expressing and/or TRA-expressing cancer stem cells in the tissue cell sample; and
a medical diagnosis or prognosis of non-aggressive and localized cancer cells in the patient based on the absence of podocalyxin-expressing and/or TRA-expressing cancer stem cells in the tissue cell sample.

2. The system of claim 1, wherein the detecting means is selected from the group consisting of immunoassays, immunostaining, immunofluorescence, immunohistochemistry, direct IHC, indirect IHC, immunocytochemistry, in situ hybridization, fluorescent ISH, FISH in suspension, western blot, flow cytometry, fluorescence-activated cell sorting, imageStream, turtle probes, target primed rolling circle PRINS, luminex assay, polymerase chain reaction, and mass spectrometry.

3. A method of determining a diagnosis or prognosis of aggressive cancer cells in a first patient having a localized tumor, comprising:
obtaining a first tissue cell sample from the localized tumor;
evaluating the first tissue cell sample for the presence of podocalyxin and/or TRA expressed on cancer stem cells;
determining a presence or an absence of podocalyxin-expressing and/or TRA-expressing cancer stem cells in the first tissue cell sample;
providing a positive medical diagnosis or prognosis of aggressive cancer cells for the first patient based on the presence of the podocalyxin-expressing and/or TRA-expressing cancer stem cells in the first tissue cell sample; and
providing a negative medical diagnosis or prognosis of aggressive cancer cells for the first patient based on the absence of the podocalyxin-expressing and/or TRA-expressing cancer stem cells in the first tissue cell sample.

4. The method of claim 3, wherein the first tissue cell sample is compared to a second tissue cell sample selected from the group consisting of an earlier obtained tissue cell sample from the first patient or a control tissue cell sample obtained from a second patient without cancer.

5. The method of claim 3, wherein the first tissue cell sample is compared to a predetermined threshold value.

6. The method of claim 4, further comprising providing a medical diagnosis of the presence of aggressive cancer cells in the first patient based on a level of the podocalyxin-expressing and/or TRA-expressing cancer stem cells in the first tissue cell sample being measurably greater than a level of podocalyxin-expressing and/or TRA-expressing cancer stem cells in the second tissue cell sample.

7. The method of claim 4, further comprising providing a medical diagnosis of the absence of aggressive cancer cells in the first patient based on a level of the podocalyxin-expressing and/or TRA-expressing cancer stem cells in the first tissue cell sample being essentially equal to or measurably less than a level of podocalyxin-expressing and/or TRA-expressing cancer stem cells in the second tissue cell sample.

8. The method of claim 5, further comprising providing a medical diagnosis of the presence of aggressive cancer cells in the first patient based on a level of the podocalyxin-expressing and/or TRA-expressing cancer stem cells in the first tissue cell sample being essentially equal to or measurably greater than the threshold value.

9. The method of claim 5, further comprising providing a medical diagnosis of the absence of aggressive cancer cells in the first patient based on a level of the podocalyxin-expressing and/or TRA-expressing cancer stem cells in the first tissue cell sample being measurably less than the threshold value.

10. A method of selecting a medical treatment for a patient having a localized tumor, comprising:
obtaining a tissue cell sample from the localized tumor;
analyzing the sample for a presence of podocalyxin-expressing and/or TRA-expressing cancer stem cells selected from the group consisting of podocalyxin-specific antibodies and/or TRA-1-60, TRA-1-81 and mixtures thereof;
determining the presence or an absence of the podocalyxin-expressing and/or TRA-expressing cancer stem cells in the sample;
providing a positive medical diagnosis or prognosis of aggressive cancer for the patient based on the presence of the podocalyxin-expressing and/or TRA-expressing cancer stem cells in the tissue cell sample; and
providing a negative medical diagnosis or prognosis of aggressive cancer for the patient based on the absence of the podocalyxin-expressing and/or TRA-expressing cancer stem cells in the tissue cell sample.

11. The method of claim 10, wherein the analyzing of the sample comprises employing a binding material selected to interact with the podcalyxin-expressing and/or TRA-expressing cancer stem cells in the sample.

12. The method of claim 11, wherein the binding material is an antibody.

13. A kit for detecting and diagnosing aggressive cancer stem cells in a patient; the kit comprising:
a tool for obtaining a tissue cell sample from the patient;
an analyzing means for determining the presence or absence of podocalyxin-expressing and/or TRA-expressing cancer stem cells in the tissue cell sample; and
an indicator means to display the presence or absence of the podocalyxin-expressing and/or TRA-expressing cancer stem cells in the sample.

14. The kit of claim 13, wherein the analyzing means comprises employing a biotinylated material selected to interact with the podocalyxin-expressing and/or TRA-expressing cancer stem cells in the sample.

15. The kit of claim 13, wherein the localized tumor is a localized prostrate tumor.
